(19) United States
(12) Patent Application Publication
(10) Pub. No.: US 2003/0008328 A1
(43) Pub. Date: Jan. 9, 2003

(54) Quantitative in vitro bone induction assay

(76) Inventors: John F. Wironen, Alachua, FL (US); Rebecca Jaw, Alachua, FL (US)

Correspondence Address: McAndrews, Held, & Malloy, Ltd., Citicorp Center, 500 West Madison Street, 34th Floor, Chicago, IL, 60661, US

(21) Appl. No.: 09/897,728

(22) Filed: Jul. 3, 2001

(51) Int. Cl. G01N 33/567; G06F 19/00; G01N 33/48; G01N 33/50; C07K 14/51

(52) U.S. Cl. 435/7.21; 702/19; 530/399

(57) ABSTRACT

An in vitro assay for quantifying the osteogenic capacity of bone implants involves in vitro isolation and quantitation of specific osteogenic factors. The method disclosed permits direct measurement of the osteogenic capacity of an implant to allow greater predictability of the degree to which new bone will grow in a given area. The method eliminates the need to practice the traditional technique of implanting material into a test animal and subsequently sacrificing the animal to assess bone growth associated with the implant. Since the present method does not involve animal testing, it is an extremely reproducible, rapid, and accurate method for predicting whether an implanted composition or material will induce bone growth without the need for in vivo assays.

### FIELD OF THE INVENTION

This invention relates generally to the isolation of osteoinductive proteins used in bone implants and specifically to an in vitro method for quantifying the osteoinductive capacity of bone morphogenic proteins isolated from bone matrices.

### BACKGROUND

Traditional methods of repairing bone damage require “setting” the bone and allowing the body's natural restorative process to repair the bone over time. For more complex fractures resulting from trauma or bone disease, metallic fixation and reinforcement devices have been employed to support the bone throughout the healing process. Often, in those situations where native bone is irreparable, bone from another source (e.g. animal, i.e. xenograft, another human, i.e. allograft, or from the same patient from a second site, i.e. autograft) is grafted to the native bone. The use of bone from allograft and xenograft resources is becoming more accepted by the public and strides are being made in alleviating concerns relating to transmission of viral and other pathogens. Toward this goal in improving current solutions for addressing bone injuries and defects, extensive research is underway in the field of osteogenesis to determine the mechanisms and agents that function in bone growth and development.

Bone morphogenesis is a continuous process in normal, healthy bone, involving complex biochemical pathways. Through cyclical processes of resorbtion and formation, bone is remodeled or repaired to meet the demands placed on the skeletal system. Briefly, the growth of bone via endochondral ossification involves: a). incursion of mesenchymal cells into the area, b). differentiation of these cells into chondroblasts or chondrocytes capable of forming cartilage, and c). migration of osteoblasts and osteoclasts into the area which progressively destroy cartilage and deposit new bone. The activities of these cellular components are regulated by hormones, growth factors and cytokines. It is now known that if osteoprogenitor cells are present at a site, bone formation may be induced through the application of osteoinductive proteins.

Bone contains multiple osteoinductive proteins including, but not limited to, transforming growth factor alpha (TGF-α), transforming growth factor beta (TGF-β) and bone morphogenic proteins (BMP-1 BMP-2, BMP-3, BMP-4, BMP-5, BMP-6, BMP-7, and others). Bone morphogenic proteins, as a group, are members of the TGF-β family of growth and differentiation factors, which function in a variety of ways to regulate cellular activity.

Bone morphogenic proteins (BMPs) are involved in a myriad of activities. In vitro studies have suggested that BMP's are likely to have significant effects on cells during several phases of endochondral bone formation in vivo. BMP's may attract cells to the implantation site via chemotaxis; they may induce progenitor cells to differentiate into cartilage-forming and bone-forming cells, and they may affect the proliferation of these cells during several phases of the bone-formation process. (Wozney, J. M. Cellular and Molecular Biology of Bone, 1993, 131-167).

In U.S. Pat. No. 6,150,328, to Wang et. al. a combination of human BMP-2 and BMP-4 resulted in the formation of cartilage-like nodules at 7 days post implantation. The amount of growth observed appeared to be dependant on the amount of BMP-2 or BMP-4 present. BMP's may also play an active role in cancer regulation. BMP-2 caused cell-cycle arrest in the G1 phase and the subsequent apoptosis of myeloma cells. (Kawamura C., et al. Blood Sep. 15, 2000;96(6):2005-11). BMP's also appear to be involved in wound healing. BMP-6 expression was upregulated after skin injury by keratinocytes at the wound edge and by fibroblasts in the wound bed (Kaiser, S. et al., 1998, J. Invest. Dermatol. 111(6):1145-52). Recent studies suggest that BMP's act as negative regulators of the development of the nervous system. Undifferentiated mouse cells lost their capacity to differentiate into neurons, but not astrocytes, after treatment with BMP-4 (Bani-Yaghoub, M. et al., Exp Neuro.l, 2000 March; 162 (1):13-26). Similarly, BMP 2, 4 and 7 were found to inhibit neurogenesis in olfactory epithelium cultures by inducing the degradation of an essential transcription factor. (Shou, J. et al. 1999, Nat Neurosci., April: 2(4): 339-45). In addition to being present in bone, BMP's are also widely distributed in non-skeletal tissues such as nerve, gastrointestinal tract, kidney, heart and lungs, and they have a central role in vertebrate and non-vertebrate organogenesis (Kirker-Head, C A., Adv Drug Deliv. Rev, Sep. 15, 2000;43 (1):65-92). For example, BMP-4 has been linked to lung development in vivo (Lebeche, D. et al., Mech Dev 1999 August, 86 (1-2):125-36). Additionally, BMP's were recently shown to regulate steroidogenesis by inhibiting ovarian androgen production in rats. (Dooley, C A, 2000, J Clin Endocrinolo Metab, September; 85(9):3331-7).

Transforming growth factors (TGF's) are multi-functional cytokines (i.e. they are pleitropic) in that they mediate a wide variety of activities: TGF-β1, for example, has been implicated as an important regulator of bone formation and resorbtion. TGF-β1 genotype affects both peak bone mass attained in adolescents and the rate of bone loss later in life, and the association of the TGF-β1 genotype with the prevalence of spinal osteoarthritis and intervertebral disc degeneration in postmenopausal women has also been noted, (Yamada Y. et al., Am J Med 106: 477-479, 1999). TGF-β1 is widely known to stimulate cell differentiation, inhibit epithelial cell proliferation and induce epithelial cell death. Recently, TGF-β1 has been linked to cancer growth. One study found that prostate cancer cells express high levels of TGF-β1 and enhance prostate cancer growth and metastasis by stimulating angiogenesis, and by inhibiting immune responses directed against tumor cells. (Wikstrom P., Scand J Urol Nephrol 2000, April;34(2):85-94). Further, TGF-β1 and integrin-mediated signaling act synergistically to enhance cell adhesion and migration and affect downstream signaling molecules of hepatocarcinoma cells. (Cai T., Chem Biophys Res Commun Aug. 2, 2000:274(2):519-25). Control of scarring in adult wounds has been reduced in response to treatment with TGF-β1. (Immunol Cell Biol 1996 April; 74(2):144-50). Additionally, TGF-β1 is known to act as anti-inflammatory agent. TGF-β1 was shown to down-regulate the inflammatory cytokine-induced expression of VCAM-1 in human glomerular endothelial cells, (Park S K et al., Nephrol Dial Transplant May 15, 2000(5):596-604).

Thus, because BMP's and TGF's are involved in a myriad of developmental and repair activities in the body, each has been the subject of a great deal of research. Since BMP's in particular appear, at least in part, to confer regenerative properties on bone, it has been the focus of much recent research directed at developing new methods of repairing damaged bone in vivo that reduce or eliminate the problems associated with healing, continued care, allograft or xenograft incompatibility, or other complications inherent in traditional bone grafting or repair.

Numerous bioabsorbable, osteogenic devices are presently available for use as implants to correct some of the problems inherent in traditional bone repair. Much of the research conducted in this area is founded on the well-known correlation that exists between new bone growth in an area and the amount of demineralized bone matrix (DBM) implanted. Thus, the current trend in the field of orthopedics has been to develop implantable materials that contain a mixture of DBM and carrier materials to help direct new bone growth. These materials have a wide range of compositions, but usually contain some form of bioabsorbable matrix, mixed with one or more materials to induce new bone formation. The material or composition is implanted into a desired location, and new bone is induced to grow in association with the implant. Alternatively, agents contained within the implant are released to stimulate new bone growth in the surrounding area. In either case, knowledge of the amount and type of osteogenic material incorporated is essential to accurately predict the likelihood that an implant will induce new bone formation.

Currently, there are no validated methods for determining the osteogenic capacity of an implant material without actually implanting the material. In vivo animal assays traditionally used to demonstrate bone growth activity of a substance are expensive and time-consuming. Existing in vitro cell-based assays have utilized methods that indirectly link osteogenic, osteoconductive, or osteoinductive implant activity with bone formation in vivo. Examples include measurement of alkaline phosphatase activity in cell cultures or proliferation of cancer cells. However, these assays have been found to show only weak correlations with in vivo bone induction in subsequent animal studies. These cell-based and animal-based assays involve large amounts of work, take weeks to months to produce results, and are inherently irreproducible because they involve living or complex biological systems. As a result, patients undergoing treatment for bone repair may face invasive procedures without any meaningful assurance that the composition of materials implanted will result in new bone growth, or bone growth to the extent needed to repair a given area. Thus, it is not uncommon for a patient to undergo duplicate invasive procedures for the same injury, despite the significant advances made in the field of bone repair. Without an easy, quantitative method for determining whether an implant will function in its desired role, primary treatment often leads to secondary hospital intervention, with the concomitant extension of recovery time and increase in costs. Thus, an in vitro method for quantifying the osteoinductive capacity of a given formulation used as an implant is needed in the field.

Several patents have issued directed at the isolation and use of bone morphogenic proteins to repair damaged bone. U.S. Pat. No. 4,608,199 to Caplan et. al., discloses a bone protein purification process, and more specifically a process for extracting and purifying soluble bone protein capable of stimulating chondrogenesis. That invention provides a process of purifying a mixture of bone matrix protein to obtain a protein capable of enhancing chondrogenesis, including fractionating a mixture of bone matrix protein, and bioassaying all fractions to identify those fractions that stimulate chondrogenic expression in undifferentiated cells in culture. The purification process is also monitored at various stages by bioassaying the bone protein for chondrogenic activity in embryonic limb bud mesenchymal cell cultures. In one example of this bioassay, chick embryo limb bud mesenchymal cells, are exposed to bone protein, and are monitored to determine if they differentiate in culture into cartilage, bone or connective tissue fibroblasts. The emergence of any of these different cell types, beyond that which is considered predictable when grown under specific conditions, was used as an indicator that substances which enhance or inhibit the limb mesenchyme-to-chondrocyte transition are present in a given fraction. However, this patent does not disclose a quantitative method for assaying the activity of bone protein, takes considerable time and uses an entirely different procedure to isolate and assay the osteoinductive activity, as compared with the present method.

U.S. Pat. No. 4,804,744 to Int. Genetic Engineering, Inc. discloses a preparation of human-derived osteogenic factors, methods for their isolation, and uses thereof to repair bone defects. The invention is directed to mammalian bone matrix-derived proteins which exhibit the ability to promote or stimulate local osteogenesis at sites of implantation in mammals. Specifically, the invention involves extraction and purification of osteogenically active protein preparations including extraction of bone matrix proteins under dissociative (denaturing) conditions, followed by further purification techniques. The bone inducing activity of various fractions was measured using a bone induction assay comprising: implantation of test material, either coated with the osteogenic preparation or not coated with the osteogenic preparation, into the calvaria of rabbits; following growth activity daily by clinical observations; removing implants at either 6 weeks or 12 weeks; removing the calvaria, fixing, decalcifying, staining and processing specimens for hematoxylin. Histomorphology and qualitative determinations of percent ossification was achieved by examination of the stained sections. Thus, the '744 method only allows for a qualitative assessment of osteogenic activity. Further, the time period to receive results is significantly different from that provided in the present invention, i.e. 6-12 weeks as opposed to less than 4 days. The '744 method requires in vivo implantation.

U.S. Pat. No. 5,169,837 to Lagarde et. al. discloses a purified osteogenic factor derived from mammalian bone that, when delivered to bone in association with a physiologically acceptable delivery vehicle, is capable of inducing new bone growth at the bone surface. The osteogenic factor is isolated from an extract of mammalian bone. In practice, bone is digested, the osteogenic factor is trapped in the soluble phase and is precipitated with ethanol. Thus, the osteogenic factor is a water-soluble component of the ethanol-precipitated bone extract. The bone formation-inducing activity of the osteogenic factor is monitored during the isolation procedure using a “rat bone growth assay”, which compares the increase in dry weight of rat bone treated with osteogenic factor, relative to an untreated contralateral bone control. Injectable solutions containing the osteogenic factor were prepared by combining a factor-containing preparation with hydroxyapatite matrix and an aqueous buffered solution, which was then delivered to the limb of a rat by single injection alongside, i.e. near the surface of, the tibia-fibula complex. A control dose, devoid of osteogenic factor, was similarly delivered to the contralateral limb. The treated and untreated bones were removed about 7 days after treatment, the bones were freed of soft tissue, washed and then dried. The increase in bone mass induced by the osteogenic factor preparation was then measured as the difference in dry weight between the treated and control bones. Depending on the amount of osteogenic factor contained in the injected preparation, a bone weight increase in excess of 25% was observed. Although the patent provides some measure of quantitative activity over a relatively short time period, it requires implantation into and extraction from an animal, resulting in the death of the subject animal. The present invention avoids the need to kill animals, while providing an expedient test that allows for rapid, precise, quantitative analysis of osteogenic activity.

Notwithstanding the variety of methods taught in the art, the references identified above do not teach or suggest an in vitro method to quantify the osteoinductive capacity of a bone implant. A need therefore remains in the field for a method to efficiently determine the osteoinductive potential of a material.

The present method uses direct measurement of growth factors responsible for bone induction to quantify the osteoinductive capacity of an implant prior to deployment in vivo. Through use of this simple in vitro assay, rapid, quantitative measurements of osteoinductive proteins extracted from bone can be achieved within about 4 days. By quantifying the amount of specific bone inductive factors incorporated into a given bone implant, the present invention allows a user to determine, prior to implantation, the likelihood that the composition will result in sufficient new bone growth to repair the damaged or diseased area. The current assay result is also highly correlative in predicting osteoinductivity of a bone sample when compared with a rat assay. The results can be expressed in a definite numerical value, thereby allowing objective quantitative standards to be developed to accept or reject tissue samples. Since the present method does not involve any living biological entities, it is extremely reproducible and eliminates the ethical and expense issues associated with live animal testing.

### BRIEF DESCRIPTION OF THE DRAWINGS

FIG. 1 is a graph depicting the relationship between BMP measured using an ELISA assay and the probability of passing an in vivo rat assay.

FIG. 2 is a table showing the estimated probability of passing an in vivo rat assay based on results of ELISA assay as compared with actual in vivo rat assay results.

FIG. 3 is a graph plotting estimated probability of passing an in vivo rat assay against the concentration of TGF*BMP.

FIG. 4 is a graph plotting observed and logistic estimate of the percent rat assays that pass osteoinductivity against the measured TGF*BMP.

FIG. 5 is a table showing the results of a logistic regression using only the product of BMP*TGF (and the intercept).

FIG. 6 is a table showing mean and standard deviation values for experimental units analyzed.

FIG. 7 is a table showing data obtained from a logistic regression of experimental units.

FIG. 8 is a first three-dimensional scatter plot graphic showing data obtained from a logistic regression.

FIG. 9 is a second three-dimensional scatter plot graphic showing data obtained from a logistic regression.

FIG. 10 is a two-dimensional scatter plot showing data obtained from a logistic regression.

FIG. 11 is a logistic regression table showing the effect of the product of BMP*TGF when added to the model.

FIG. 12 is a first three-dimensional contour plot showing the effect of BMP*TGF added to the model.

FIG. 13 is a second three-dimensional contour plot showing the effect of BMP*TGF added to the model.

FIG. 14 is a two-dimensional contour plot showing the effect of BMP*TGF when added to model.

FIG. 15 is a table showing the results of an estimate of regression analysis for BMP*TGF only.

FIG. 16 is a first three-dimensional contour plot showing estimates of BMP*TGF use only.

FIG. 17 is a second three-dimensional contour plot showing estimates of BMP*TGF use only.

FIG. 18 is a third three-dimensional contour plot showing estimates of BMP*TGF use only.

FIG. 19 a line graph of a logistic regression (logit).

### SUMMARY OF THE INVENTION

This invention is an in vitro method for quantifying the osteoinductive capacity of bone implants. Bone inductive proteins are isolated from bone matrix, and quantified in vitro prior to implantation to assess the osteogenic capacity of a given composition. The composition may be subsequently used to generate bone at a site where skeletal tissue is deficient due to injury or disease. The method allows direct measurement of the amount of bone inductive factors present in an implant and thus allows greater predictability of the degree to which new bone will grow in a given area upon implantation. Furthermore, the analytical method of this invention takes less than four days to complete, does not involve animal testing and is extremely reproducible.

Accordingly, it is one object of this invention to provide a method of quantifying the osteoinductive capacity of a bone implant.

It is a further object of this invention to provide a method of measuring the regenerative capacity of a bone implant.

It is a further object of this invention to provide a method of quantifying the chondrogenic capacity of a bone implant.

It is a further object of this invention to provide a method of accelerating wound healing and the rate of recovery from bone damage or disease.

It is a further object of this invention to provide a method of stopping, reducing or preventing degenerative bone disease.

It is a further object of this invention to provide a method for the early detection of bone cancer.

It is a further object of this invention to provide a method for assessing developmental disorders associated with cell proliferation, apoptosis, differentiation and morphogenesis.

It is yet a further object of this invention to provide a method for reducing the need to test and sacrifice laboratory animals used in bone growth studies.

Other objects and advantages of this invention will become apparent from a review of the complete disclosure and the claims appended to the disclosure.

### DETAILED DESCRIPTION OF THE INVENTION

The present invention provides a quantitative, reproducible, rapid, in vitro method for determining the osteogenic potential of a sample without the need for implantation in vivo or use of biological systems. The method generally applies to any sample which may be compatible with human or non-human applications in which the implant itself is thought to have a degree of osteogenic potential.

As used herein, the term osteogenic potential is intended to imply the degree to which an implant will stimulate the production of new bone formation upon implantation into a human or non-human vertebrate recipient's tissue in vivo. Thus, the term osteogenic potential is used interchangeably with the term osteoinductive potential herein.

As used herein, the term “implant” is intended to imply any material which is non-toxic and compatible with human or non-human vertebrate tissues, and which is useful in the correction, repair, augmentation, or alteration of bone structures in the human or non-human recipient of the implant. In one principal embodiment of the invention, the implant is an implant composed substantially of mineralized or demineralized cortical bone, cancellous bone, or cortical-cancellous bone, whether in the form of an autograft, allograft or xenograft, as these terms are understood in the art. Thus, in one embodiment of this invention, the in vitro assay of this invention is utilized to determine the concentration of growth factors (e.g., BMP or TGF-beta) of a spinal implant composed substantially of cortical bone, such as that disclosed and claimed in U.S. Pat. Nos. 5,814,084, 6,096,081, or 4,950,296, each of which is hereby incorporated by reference for this purpose. In another embodiment according to this invention, the in vitro assay of this invention is utilized to determine the concentration of factors in a substantially ceramic implant used in the augmentation or correction of a maxillofacial defect. In a further embodiment of this invention, the in vitro osteogenic potential assay method of this invention is utilized to confirm that a metallic implant infused with osteoinductive factors will in fact induce the formation of new bone upon implantation of the metallic implant. In yet a further embodiment according to this invention, the in vitro osteogenic potential assay of this invention is utilized to confirm that an implant which is intended to have no osteogenic potential is in fact substantially devoid of osteogenic potential, such that upon implantation, unwanted bone formation at a particular site of implantation does not occur. Further embodiments, aspects and utilities of this invention will become apparent to those skilled in the art from a review of the complete disclosure herein and the claims appended hereto.

In essential form, the in vitro osteogenic potential assay method of this invention involves the extraction of osteogenic factors included in, adsorbed to, infused within, adhered to or in any other way associated with an implant prior to implantation thereof into a human or non-human recipient. The material extracted from an implant which contains the osteogenic factors therein is referred to herein as the “implant releasate”. Quantitative extraction of relevant osteogenic factors present in the implant releasate and accurate reflection of the total content thereof per unit mass of the implant is a further significant aspect of the present invention. Thus, in one embodiment of the invention, an implant composed substantially of cortical bone is demineralized, followed by dissolution of the residual collagenous bone matrix to quantitatively liberate osteogenic factors into the implant releasate, followed by elimination of any potentially interfering debris, and quantitation of released osteogenic factors, all without the need for in vivo implantation of the implant. As defined herein, the quantitative determination of osteogenic factors in the implant releasate is sufficient to establish the in vivo osteogenic potential of the implant composed substantially of cortical bone upon implantation thereof into a recipient.

Naturally, those skilled in the art will appreciate that in order for this invention to be of utility in a given application, it is necessary for there to be a collection of substantially identical implants available, the osteogenic potential of which is to be determined. By randomly sampling a statistically significant representative number of such implants, it is possible to establish within statistically significant parameters what the osteogenic potential of any member of the inventory is likely to be upon in vivo implantation of non-assayed implants. This is particularly significant where the embodiment of the assay of this invention results in total destruction of a given implant upon assay. It will be appreciated that for certain types of implants, it is possible to apply the method of this invention and still utilize the implant assayed. Thus, for example, it is possible to test a metallic implant for osteogenic potential due to infusion or adhesion of osteogenic factors therein or thereto. Subsequent to assay, the metallic implant may be once again infused or adhered with osteogenic factors, and the same implant that was assayed may then be implanted, with an assurance of the known level of osteogenic potential, so long as the infused or adhered osteogenic material itself is uniform in nature and does not alter in activity during initial application and application subsequent to assay. Naturally, the method of this invention may be applied to a plurality of osteogenic factors directly, in a suitable dilution series as necessary, to determine the osteogenic potential of the infusate or adherent composition which is to be applied to or infused into an implant. Those skilled in the art are well familiar with the desirability of establishing internal standards and linear assay ranges in biological test systems, such that great detail is not provided herein in order to enable those skilled in the art to practice this aspect of the invention.

Turning now to various specific embodiments of the present method, in one embodiment, bone matrix is subjected to demineralization according to methods known in the art (such as, but not limited to acid extraction of bone minerals, use of chelating agents such as EDTA, and the like). The residual bone structure upon demineralization of bone is substantially a collagenous matrix, within which bone inductive factors are trapped.

Dissolution of the collagenous matrix by means known in the art or means hereafter developed are applied to liberate the bone inductive factors. A method of demineralizing bone and isolating osteoinductive proteins is discussed by Jortikka et. al., Ann. Chir Gynaecol Suppl, 1993, and is incorporated by reference herein for this purpose. Thus, the collagenous matrix in one embodiment of the invention is contacted with proteolytic enzymes which do not destroy the bone inductive factors. Such enzymes include, but are not limited to, collagenases known in the art, which do not destroy bone morphogenetic proteins, chondrogenic proteins, tissue growth factors and the like. Those skilled in the art will further appreciate that, in order to be active, certain proteolytic enzymes require the presence of buffer solutions, salt solutions, cofactors and the like. Those skilled in the art of protein chemistry, and in particular the advanced art of collagen protein chemistry, are well skilled in the options available for collagenous matrix dissolution without the need to disrupt osteogenic factors. The material remaining after dissolution of the collagenous matrix is treated to remove materials which might interfere, inhibit, or otherwise adversely affect subsequent quantitation of released osteoinductive factors. Where a method of quantitation of osteoinductive factors is chosen which is impervious to such interference, direct quantitation of released factors at this stage is acceptable. Thus, for example, where it is determined that a radio-immunoassay (RIA) is not adversely affected by the presence of collagenase and collagen degradation products, then direct quantitation of osteoinductive factors by RIA at this stage is completely acceptable, and comes within the scope of the present invention. Likewise, enzyme-linked immunoadsorbent assays (ELISAs) known in the art, immunoprecipitation assays, and the like may interchangeably be applied according to this invention at this stage, provided that it is determined that interfering materials do not destroy the accuracy and precision of the quantitative detection method chosen.

Where it is determined that interfering materials remain upon release of the osteogenic factors from the implant, these factors are removed by any of a number of standard methods known in the art which do not remove the osteogenic factors from the implant releasate. Thus, for example, the implant releasate may be centrifuged at a speed sufficient to remove debris which minimally associates with osteogenic factors. Thus, for example, the implant releasate may be centrifuged at between approximately 5,000 RPM and 15,000 RPM. The supernatant containing digested bone material is then directly quantitated for osteogenic factors, or is further treated to remove potentially interfering substances. Thus, for example, not meant to be limiting, the implant releasate supernatant may be dialyzed, ultrafiltered, precipitated, affinity purified, size fractionated by size-exclusion chromatography, desalted by mini-desalting column gel permeation, high-performance liquid chromatographic separations, or otherwise treated according to methods known in the art to remove small molecules from the larger molecular mass osteogenic factors. In light of the present disclosure and guidance provided herein, those skilled in the art are well able to select various methods for specific implant releasates to ensure that subsequent quantitation is not interfered with by non-osteogenic factor implant releasate materials, while at the same time, quantitatively retaining the osteogenic factors to be assayed. The specific osteoinductive proteins, peptides or other factors contained in the solution are then detected in picogram to milligram quantities.

The entire procedure according to the present invention may take between a couple of hours to about three to four days, depending on the number of steps required and the assay methodology utilized. In one embodiment according to the present invention, an ELISA assay is used to identify specific bone inductive proteins. Proteins of interest include, but are not limited to selected bone morphogenetic proteins, tissue growth factors, fibroblast growth factors, platelet derived growth factors, vascular endothelial growth factors, cartilage derived morphogenetic proteins, insulin-like growth factors, and the like and combinations thereof. Thus, in one specific embodiment according to this invention, BMP-2/4 and TGF-β1, for example, are liberated from a demineralized implant comprising cortical allograft bone. The BMP-2/4 and TGF-β1 are quantified using ELISA or RIA or like methods commercially available and known in the art, subsequent to quantitative release thereof and retention thereof in an implant releasate fraction to be quantitated. Thus, briefly, according to this exemplary embodiment of the invention, implant releasate fractions and dilutions thereof are coated onto wells of a microtiter plate. In a separate series of wells of the microtiter plate, known standard dilutions of BMP-2/4 and TGF-β1 are also adhered. A blocking buffer is applied to the wells to prevent subsequent nonspecific binding of antibodies to the plastic wells. BMP-2/4 and TGF-β1 specific antibodies are contacted with the bound BMP-2/4 and TGF-β1. The BMP-2/4 and TGF-β1 antibodies themselves may be labeled, radioactively, chemiluminescently, enzymatically or the like, in which case unbound antibody is washed away, and bound antibody is quantitated to provide a quantitative measure of the amount of osteogenic factor bound to each microtiter well. Alternatively, a second antibody which specifically binds only to bound first antibody may be contacted with each microtiter well. If the second antibody is labeled with a detectable label, the signal of bound osteogenic factor may be significantly amplified through use of the second antibody, whether enzyme-linked, radioisotopically labeled, chemiluminescently labeled or the like. Where an enzyme-linked immunosorbent assay is utilized as the detection method of choice, to determine the degree of specific antibody binding to osteoinductive factors, a suitable substrate for the enzyme-linked antibody complex is added to the microtiter wells. The enzyme-substrate reaction generates an end product with either color, fluorescent, chemiluminescent, or radioactive properties. The amount of end product measurable by color intensity, radioactivity or like detectable label is proportional to the amount of specific antibody binding. Through use of standard curves established by use of known quantities of osteogenic factors being detected, the degree of label detected is directly convertible to a measure of osteogenic factor present in the implant releasate samples. There are several advantages to using an ELISA assay as a screening method for clinical bone samples, the most important being that the ELISA result is highly correlative in predicting osteoinductivity of a bone sample when compared with a rat assay. The ELISA result can be expressed in a definite numerical value allowing development of quantitative standards for use in acceptance or rejection of a bone sample on the basis of the ELISA procedure. For example, results obtained from an assay may be used to generate cut-off points for the content of the BMP-2/4 and TGF-β1 in DBM for the determination of osteoinductivity.

In an alternative embodiment, a composition comprising both a mitogen and a morphogen in a carrier is produced. Preferably, the composition is engineered such that the mitogen is released first followed by the release of the morphogen. The inventors believe that this sequential release will enhance the efficacy of the composition, as the mitogen would act to increase the population of available cells for morphogenesis. In a specific example, the composition comprises TGF-beta as the mitogen and BMP-2 as the morphogen. The subject composition can be engineered by techniques and materials well-known in the art to effectuate the sequential release of the mitogen and morphogen. Furthermore, one or more of each can be included in the composition.

Those skilled in the art will recognize that there are multiple complexes and formats available for antibody detection of osteogenic factors present in implant releasates, and the invention is not limited to the specifics of the examples provided herein. This exemplary support is provided purely for purposes of providing a complete written description of the methods by which the invention may be practiced and utilized, including the best mode thereof. Thus, the following examples are intended to further illustrate, but not limit the invention.

### EXAMPLE 1

### Correlation of the in vitro Quantitative Analysis of BMP-2/4 and TGF-β1 in DBM with the in vivo Osteoinductivty of DBM Samples Used in a Rat Assay

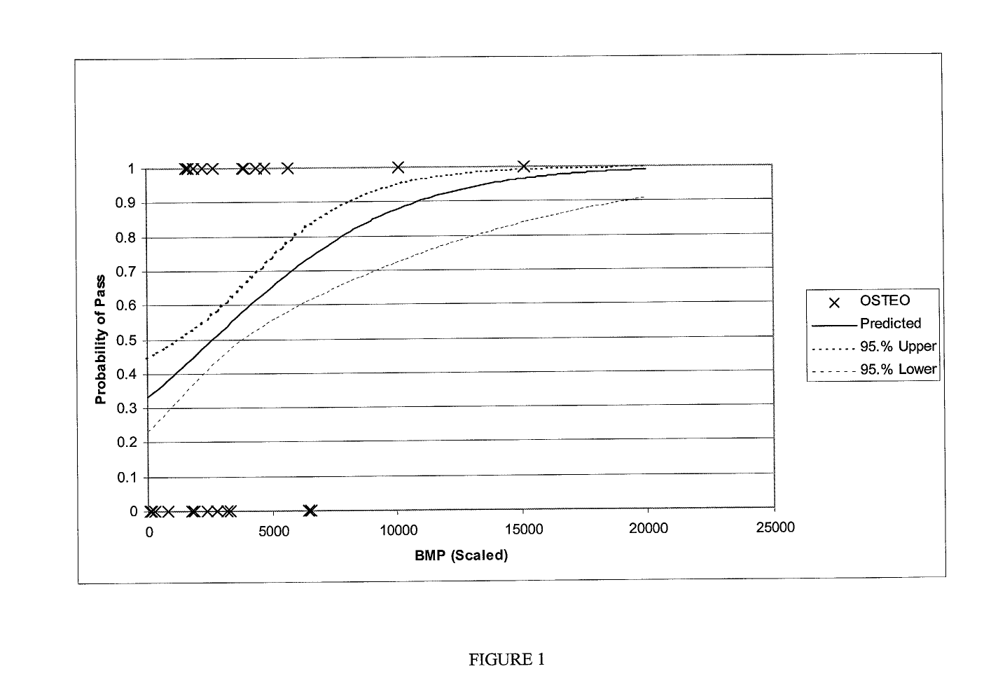

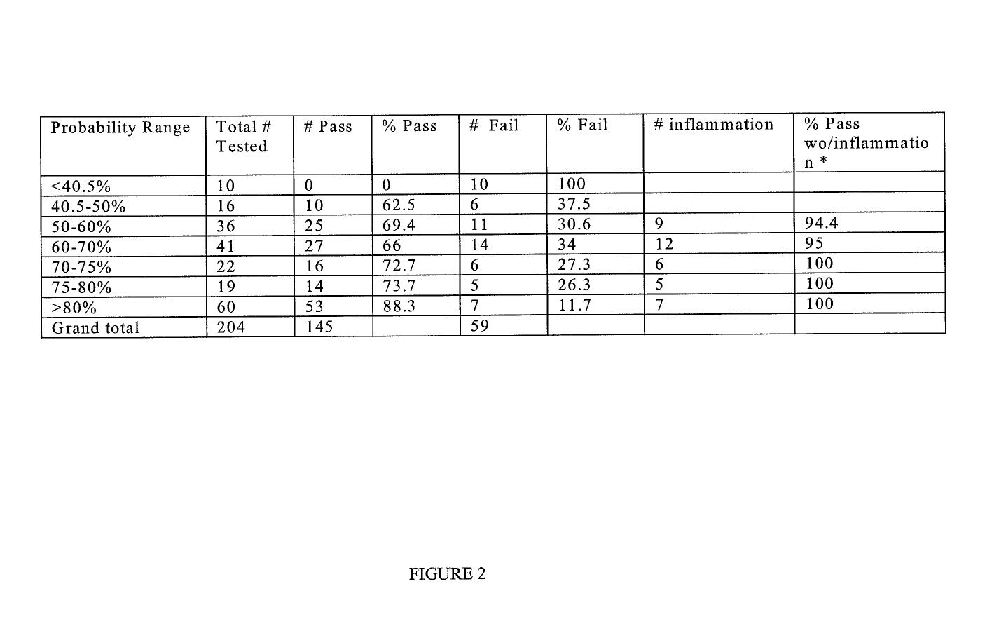

Human cortical bone was ground into a powder using a proprietary mill and then demineralized by agitation in cold (4° C.) 0.5 N HCl until the calcium content was less than 3%. The DBM was then lyophilized. A 0.4 g sample of the lyophilized DBM was digested with Type 1 collagenase in a neutral Tris-buffer. The supernatant was dialyzed against 5 mM Glutamic acid buffer. A precipitate formed inside the dialysis bag was collected by centrifugation and was dissolved in a 2M guanidine hydrochloride solution adjusted to pH 7.2 with 0.25 M EDTA. The resulting suspension was analyzed for BMP-2/4 and TGF-β1 within approximately three to four days total assay time using commercially available ELISA kits purchased from R&D Systems. In vivo osteoinductivity tests were carried in athymic nude rats according to the ectopic assay as described by Urist (1965). Bone formation of DBM samples implanted into rats was quantified after 4 weeks using a histologic scale of scores from 0 to 4 where 2 and above were rated as pass. FIG. 1 is a graph showing observed passes and failures of osteoinductivity, “osteo” (passes represented by an x placed on 1, failures represented by an x placed on 0) plotted against the measured BMP (Scaled) (predictions and bounds were approximated by asymptotic methods based on a hypothetical sample size of 92). The correlation between in vivo and in vitro results was then evaluated by statistical analysis. FIG. 2 shows the estimated probability of passing or failing a DBM sample on the basis of the amount of BMP-2, 4 and TGF-β1 present as determined by the ELISA method, as compared with in vivo rat assay. Samples that induced inflammation of 3+ were eliminated from the data set. Ignoring the samples compromised by inflammation, the correlation between in vitro ELISA and in vivo rat assay results is evident from the last column of the table in FIG. 2. As shown, a 94.4% and 95% correlation existed within the range of probabilities of 50-60% and 60-70%, respectively. Furthermore, at probability ranges higher than 70 the correlation was 100%. Standard curves showed that the assays were linear between 0.03 and 1.0 ng/ml for BMP-2/4, 2.0 ng/ml for TGF-β1. The results obtained upon repeat analysis from a single sample were statistically reproducible, with a standard deviation of ±0.01 for BMP-2/4 and ±3.2 for TGF-β1.

### EXAMPLE 2

### Correlation Between the Estimated Probability of Passing the Rat Assay vs. BMP-2/4 and TGF-β1 Product Concentration Derived from the in vitro ELISA Test

Values of BMP-2/4 and TGF-β1 were converted to a logistic estimate of percentage passing (p) using the formula: p=100(1/(1+e-(-0.5353+(3.103x10-2)[BMP][TGF])%

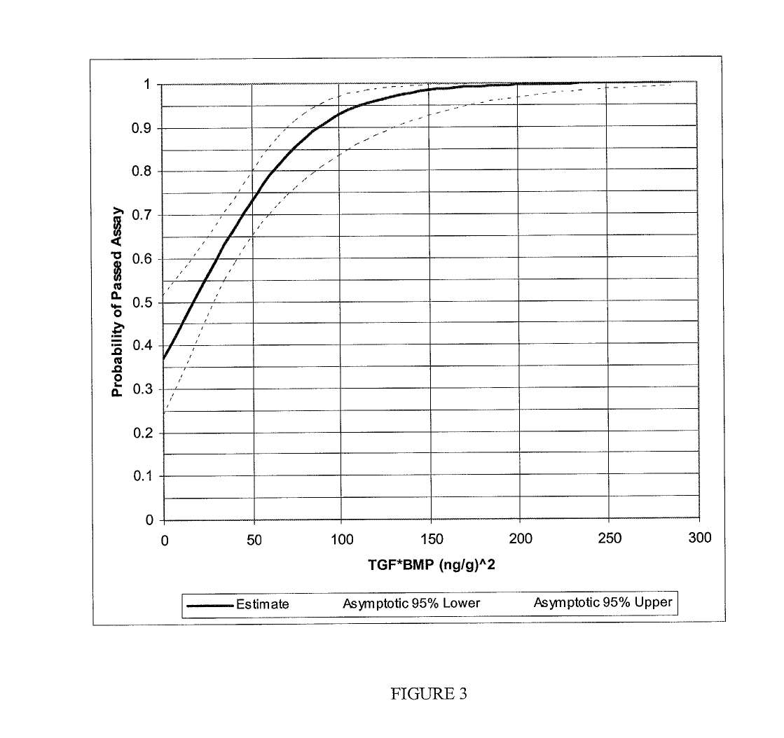

FIG. 3 graphically represents the estimated probability of passing a rat assay vs. the product of TGF and BMP concentration derived from the in vitro ELISA assay. A positive correlation was observed between the increase in the product of TGF and BMP [TGF*BMP] and the increase in probability of an implant passing an in vivo rat assay test. The 95% asymptotic confidence interval also indicates a significant correlation between TGF*BMP and the probability of passing an in vivo rat assay.

### EXAMPLE 3

### Correlation of Osteoinductivity with Increased Concentration of BMP*TGF β-1

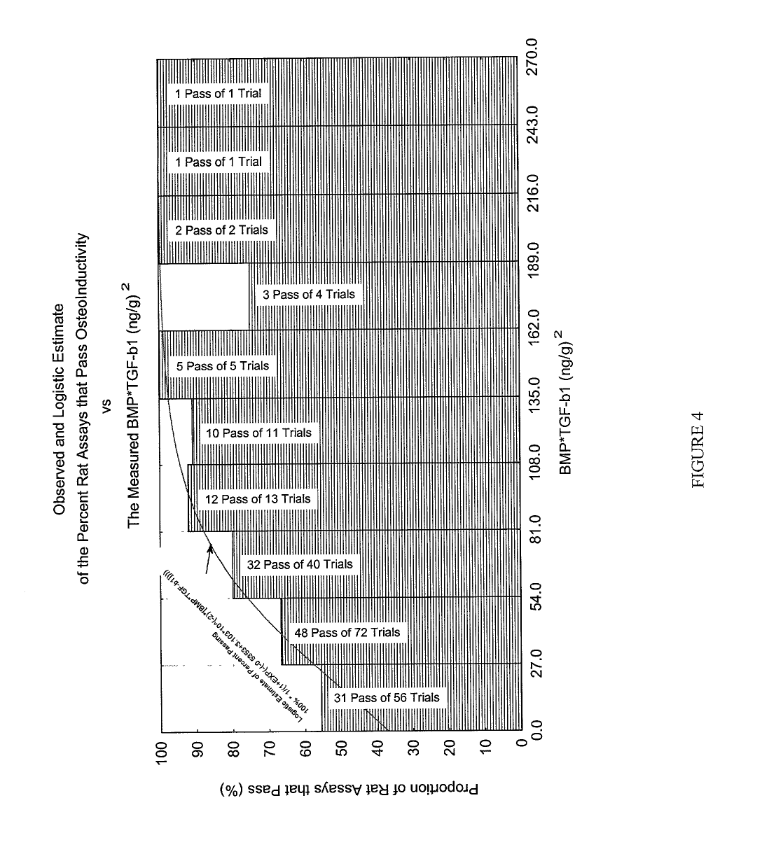

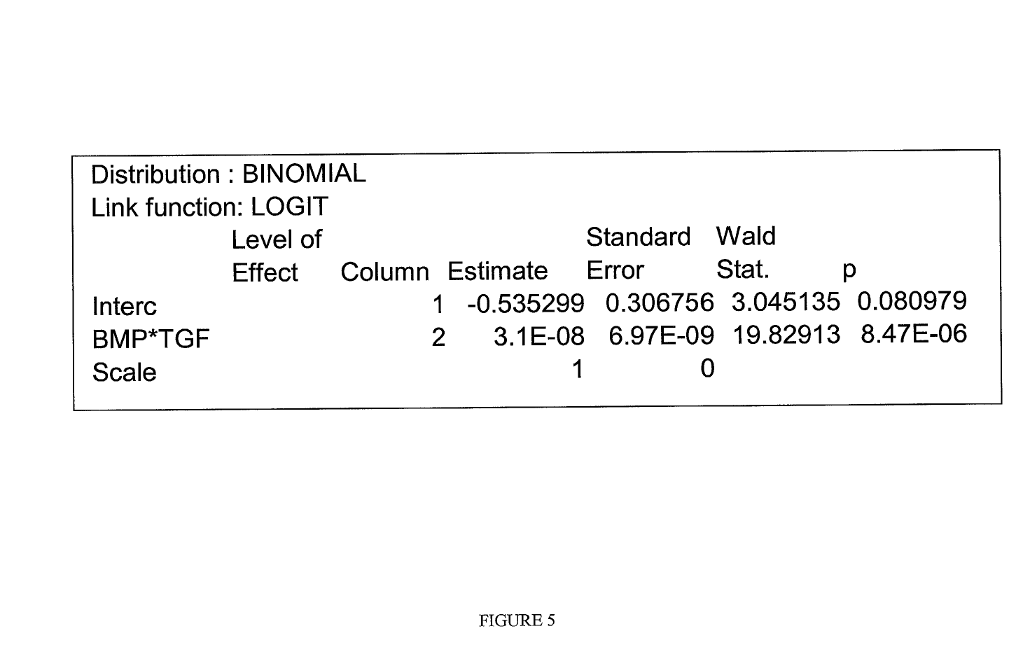

FIG. 4 graphically illustrates the increase in passed in vivo rat assays for increased BMP*TGF β-1 concentrations. As the content of the growth factors BMP*TGF-β1 (ng/g)² increased from <27 to 81 the osteoinductivity also correspondingly increased from about 40% to approximately 92%. Furthermore, as (BMP*TGF-β1) increased to 135 and up to 270 the osteoinductivity reached a plateau at 100%. In the range of 162 to 189, there were 3 samples that showed inflammation (score 3+) which may account for the 25% drop in rat assay. The line graph superimposes the estimated probability using data obtained from a logistic regression likelihood ratio test with parameter estimates. Results of regression analyses used to create the line for this test are provided in the table of FIG. 5. These data clearly demonstrate that the amount of BMP-2/4 and TGF β1 as detected by ELISA correlates with the in vivo osteoinductivity of the rat assay of a particular DBM sample.

### EXAMPLE 4

### Multiple Logistic Regression Model for Determining the Probability of Passing or Failing a DBM Sample on the Basis of the Amount of BMP-2, 4 and TGF-β1 Present as Determined by the ELISA Method

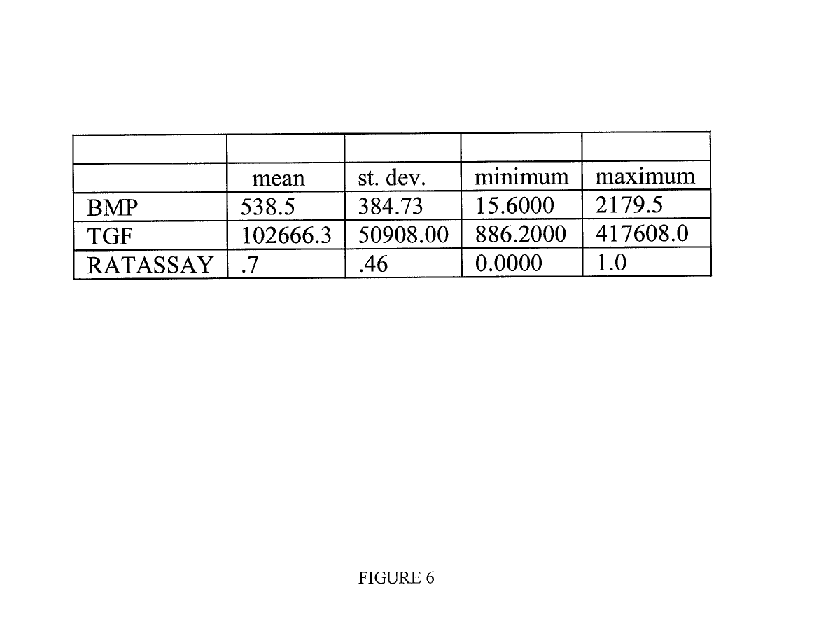

Samples (n=193) were tested for TGF and BMP concentrations and samples were analyzed to determine whether the sample passed or failed (note: re-tests without a final result were treated as fail) the assay. The number of valid cases, which passed the rat assay was 134 and the number of valid cases which failed the rat assay was 59. FIG. 6 depicts a data table comprising mean and standard deviation values for all data collected. A multiple logistic regression model was established wherein the dependent variable was considered to be the probability of rat assay pass: Pᵢ i.e. (Pass=1, Fail=0), and the regressors were considered to be TGF and BMP according to the equation:

ln(Pᵢ/(1−Pᵢ))=Constant+a*TGF+b*BMP

(Where a and b are coefficients of TGF and BMP).

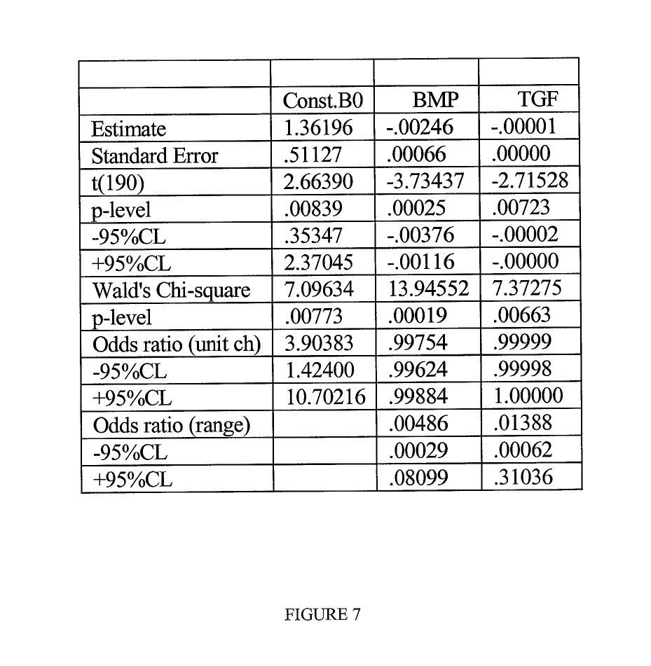

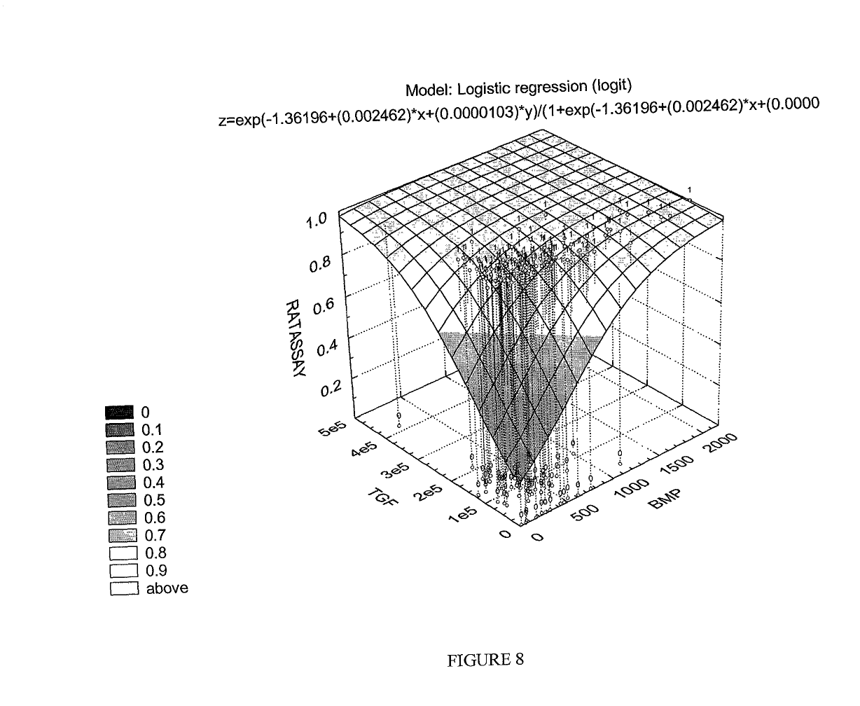

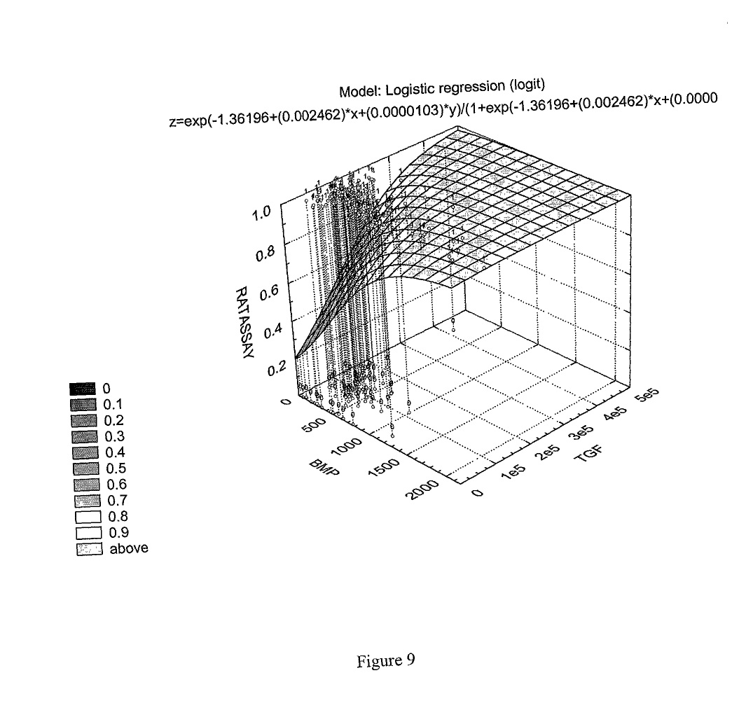

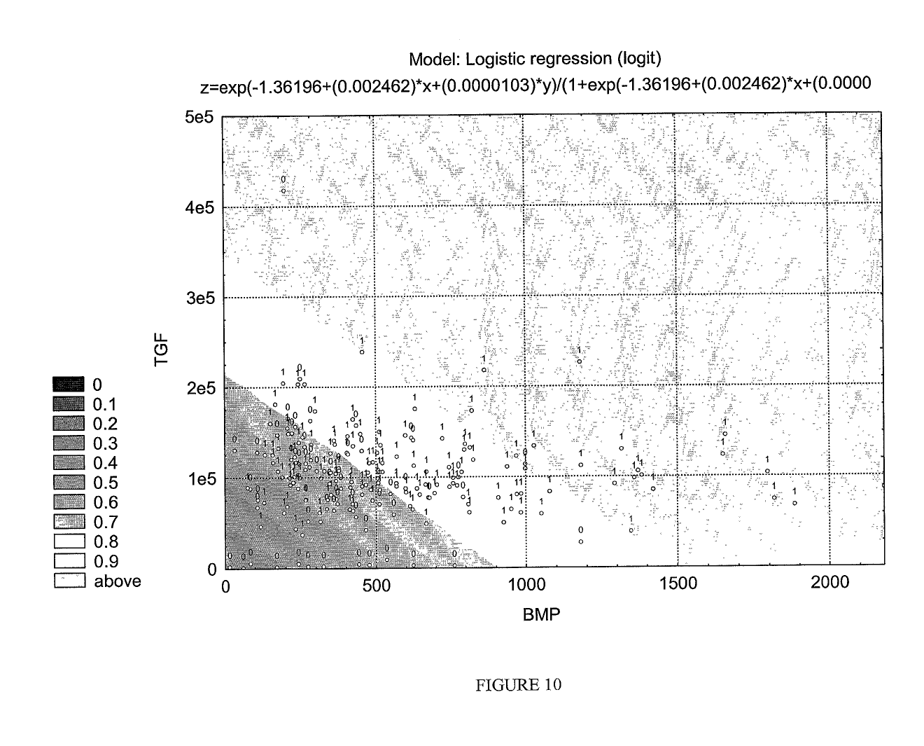

FIG. 7 shows data obtained from a logistic regression (logit) analysis which indicates a significant effect of TGF and BMP on the probability of passing a rat assay. FIGS. 8, 9 and 10 are scatter plots of estimates of regression wherein the point estimate of the probability Pᵢ of a rat assay passing was given by the formula:

Pᵢ=Pᵢ′/(1−Pᵢ′)

Where: Pᵢ′=exp(−1.3620+BMPᵢ*2.462*10⁻³+TGFᵢ*1.026*10⁻⁵)

(Note: Pᵢ′ is the point estimate of the odds ratio for a rat assay passing).

### EXAMPLE 5

### Multiple Logistic Regression Model Showing Interaction Between BMP-2, 4 and TGF-β1 when Added to the Model

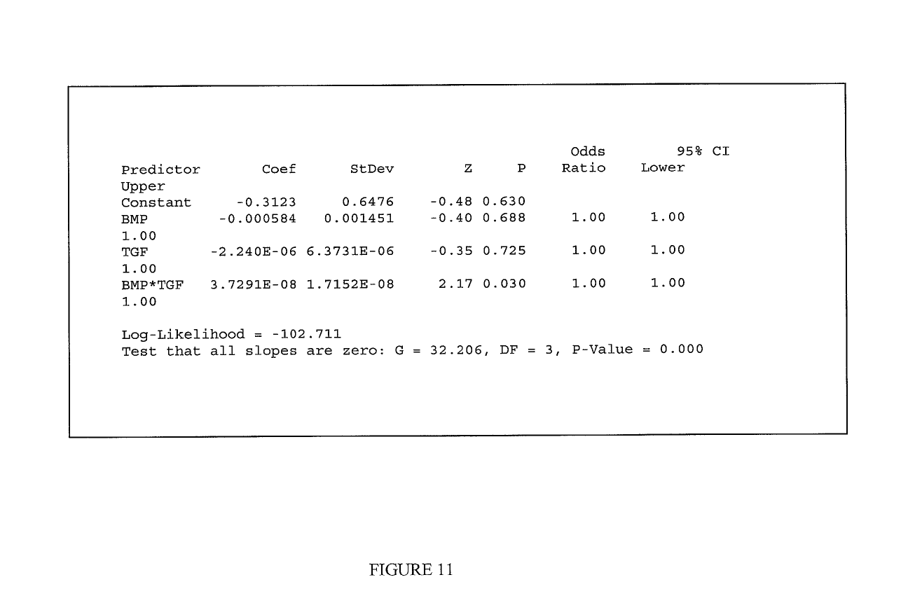

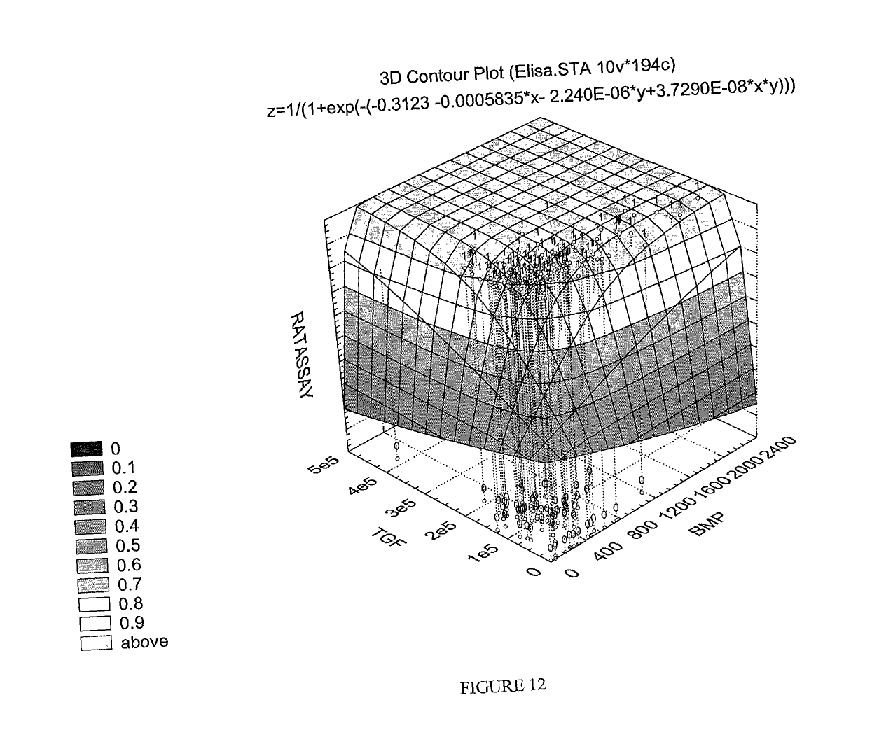

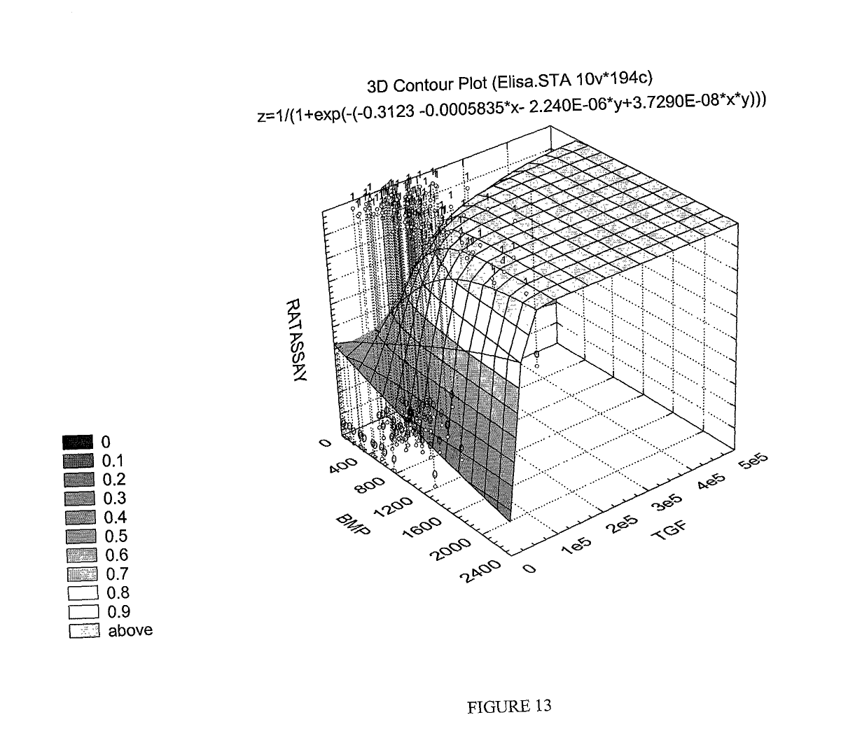

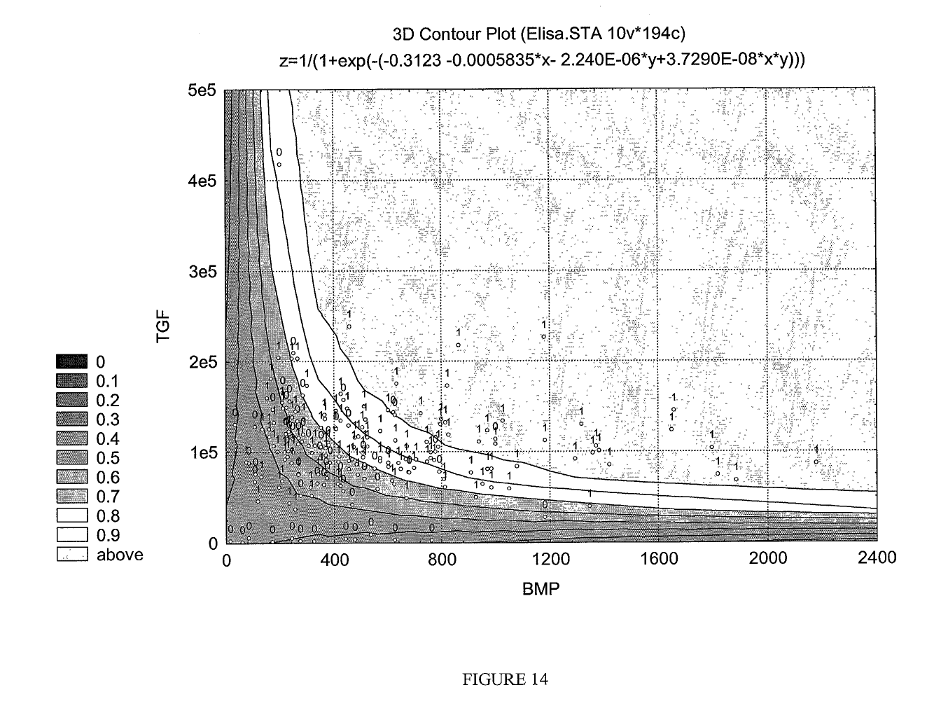

To test for an interaction between BMP and TGF, the product of BMP and TGF levels were multiplied and added to the model described in example 4. FIG. 11 shows a table comprising results obtained from a logistic regression analysis. This table indicates a significant effect of the product of BMP*TGF when added to the model containing TGF and BMP. FIGS. 12, 13 and 14 are scatter plots of data obtained on product interaction using the model previously described. The results indicated that there was a significant interaction between BMP and TGF when used as a product (BMP*TGF β1) and that the product was a significant factor when associated with other factors. When the product was used in the model, the main effects of BMP and TGF on the model were no longer significant. That is, the product was more correlative than TGF+BMP or either alone.

### EXAMPLE 6

### Multiple Logistic Regression Model Using Only the Product of BMP-2/4 and TGF-β1 and Probability of Passing or Failing a DBM Sample on the Basis of the Amount of BMP-2/4 and TGF-β1 as Determined by the ELISA Method

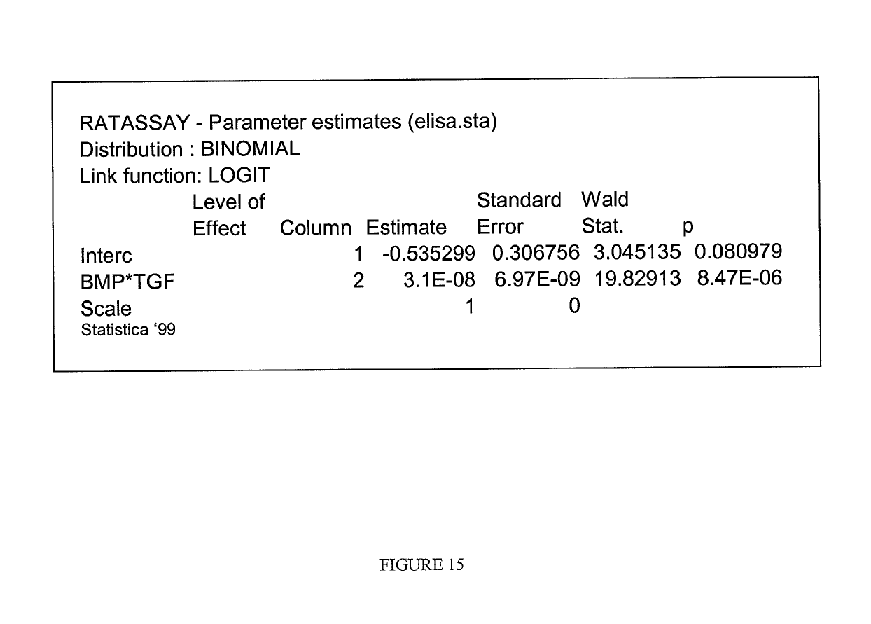

FIG. 15 shows results obtained from a regression analysis using only the product of BMP*TGF β1 (and the intercept). The estimated function using the product of BMP*TGFβ1 was described using the equation:

Pᵢ=Pᵢ′/(1−Pᵢ′)

(where Pᵢ′=exp(−0.535308+(3.10276e−8)*TMP*BMP);

and: Pᵢ′ represents an odds ratio)

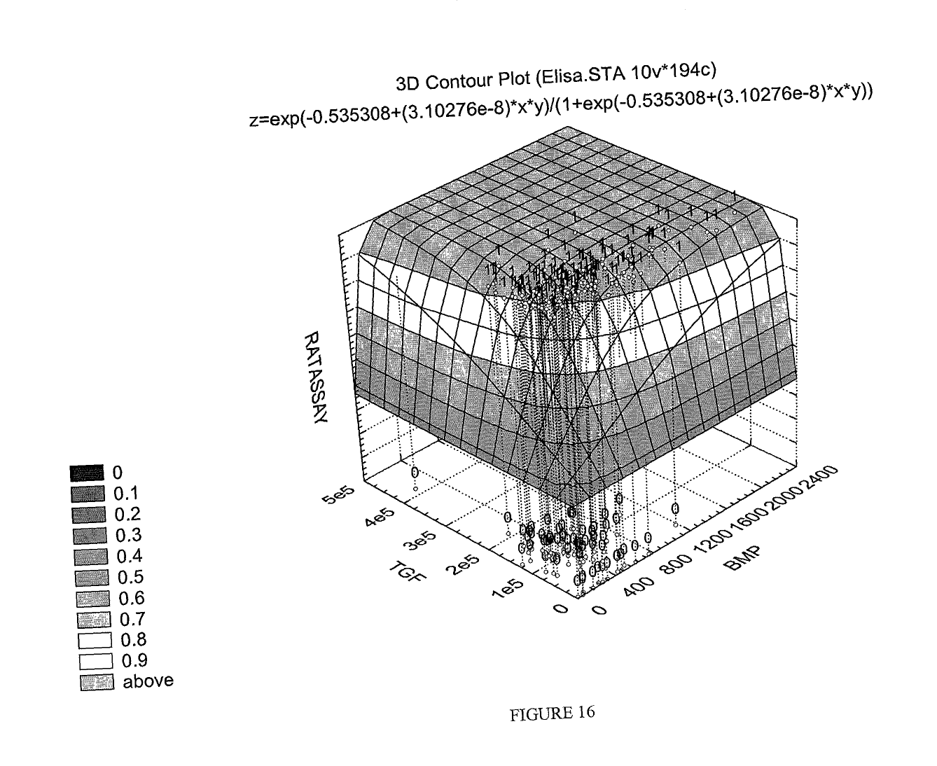

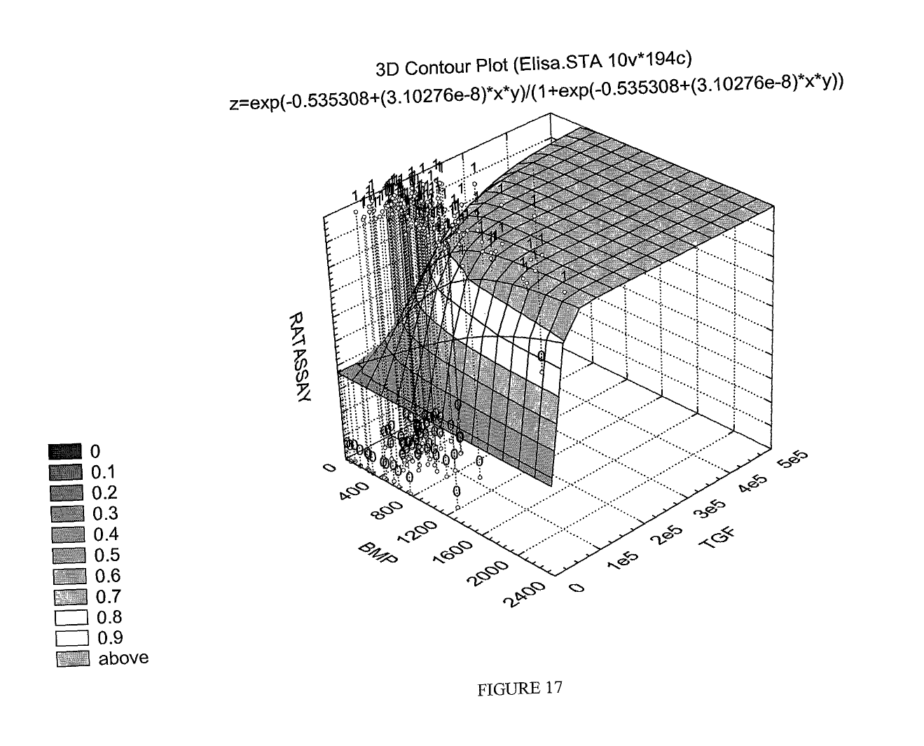

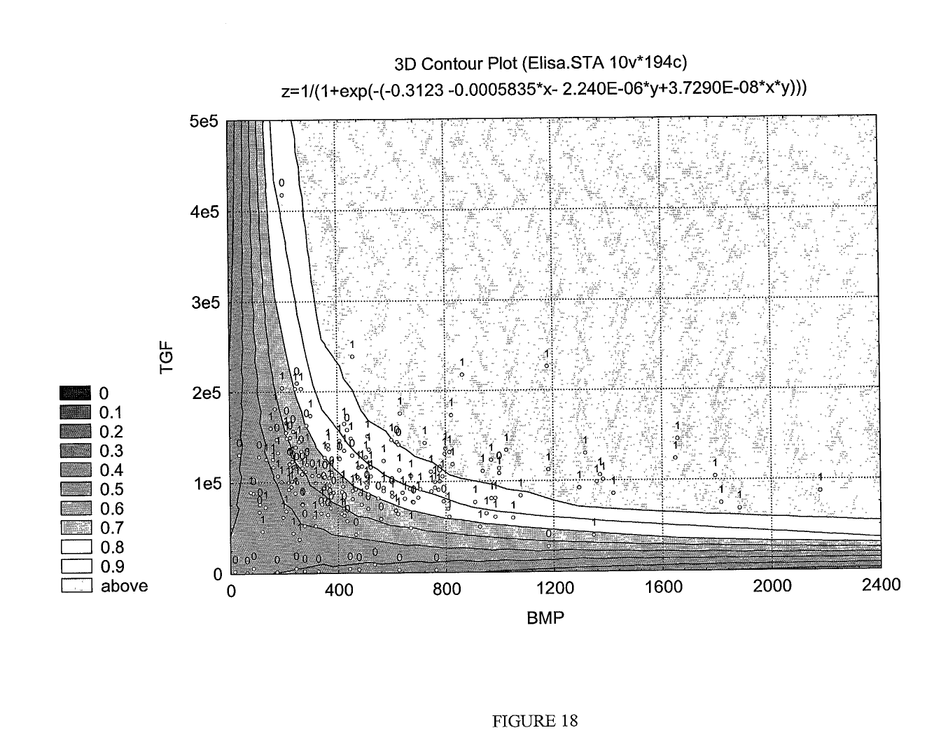

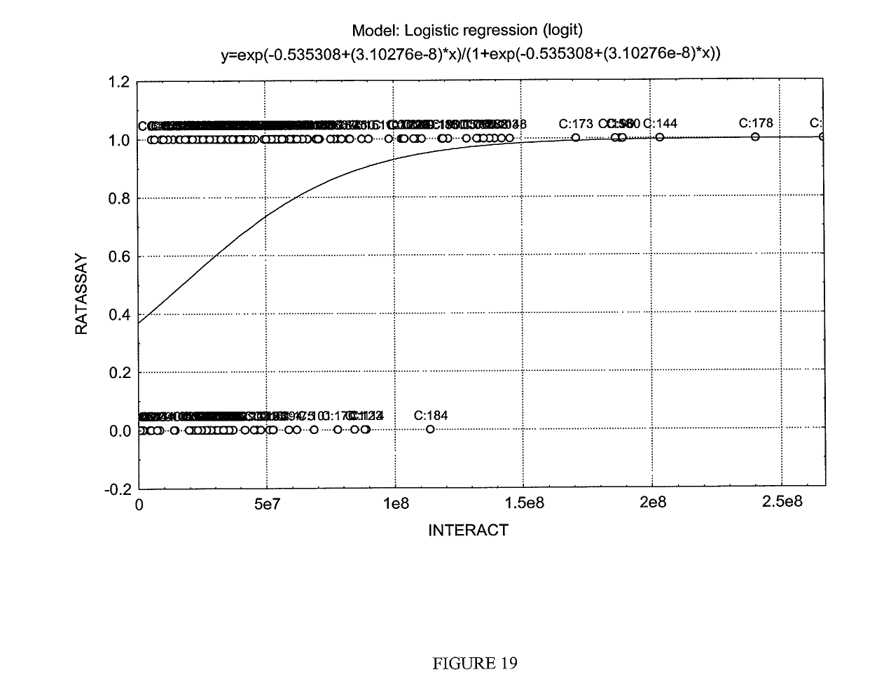

FIGS. 16, 17, 18 and 19 show graphs of estimates using only the product BMP*TGF β1 (with an intercept). Point estimates under the model are depicted. A comparison of the results of grafts using only the product with the model containing TGF and BMP as well as their product did not show obvious difference in the results. This data indicates that BMP*TGF β1 is the only statistically significant factor in the model and may be an adequate predictor of osteoinductivity thereby eliminating the need to use the additive components of BMP and TGF.

### EXAMPLE 7

### Method for Quantifying the Osteoinductive Capacity of an Inventory of Implants

The osteoinductive capacity of a statistically significant sample of implants from a collection of similar or identical implants is quantified by isolating and purifying osteoinductive proteins from implant releasate. The quantitated osteoinductive factor is selected from the group consisting of bone morphogenetic proteins, tissue growth factors, fibroblast growth factors, platelet derived growth factors, vascular endothelial growth factors, cartilage derived morphogenetic proteins, insulin-like growth factors, and the like and combinations thereof. The assay is conducted in the presence of known standard titrations of the osteoinductive or chondrogenic factor being quantitated and a standard curve is established for determining absolute concentrations of the quantitated factors from implant releasate. A determination of statistical significance of any deviations from a mean osteoinductive potential measurement for a given implant selected from the inventory is calculated to provide a measure of osteoinductive potential for the entire inventory of similar or substantially identical implants in the inventory. To be certain that there is a good correlation between the osteoinductive potential measured according to the wholly in vitro method of the present invention and action in vivo upon implantation of implants selected from the assayed inventory, a representative sampling of implants are implanted in vivo and measured according to standard methods known in the art for determining osteoinductivity. Thus, for example, not meant to be limiting, methods disclosed, referred to or suggested in U.S. Pat. No. 6,189,537, hereby incorporated by reference for this purpose, may be used to confirm that the in vitro osteoinductive potential measured according to the present invention correlates well with in vivo bone induction. In this manner, an inventory of implant materials may be quality controlled for osteoinductive potential with a high degree of confidence that the specific conditions for measuring osteogenic potential according to the in vitro methodology of this invention provides consistently reliable results when extended to in vivo implantation. By following this methodology, those skilled in the art are enabled to select specific combinations of osteogenic factors to quantify in vitro and determine a correlation factor for prediction of in vivo osteogenic potential. Thus, for a specific application, it is determined that it is sufficient to measure in vitro only the level of BMP-2/4 and TGF-β1 present in an implant to predict with a high degree of confidence what level of osteogenic activity is likely to be achieved upon implantation in vivo. In another case, it is determined that it is critical to measure both the concentration and total amount of BMP-2/4 and TGF-β1 present in an implant. In a further embodiment of the invention, it is determined that it is sufficient to measure only the level of TGF present in an implant, while in yet a further embodiment of the invention, a combination of multiple osteogenic factors is measured in order to acquire a consistent, reproducible, accurate and precise measure of ultimate in vivo osteogenic potential.

### EXAMPLE 8

### Measurement of the Osteoinductive Capacity of a Composition Used with Implants

The regenerative and osteoinductive capacity of a composition for use in combination with an implant, by infusion therein, coating or adhesion thereto, is measured in vitro according to the method of the present invention. Thereafter, known quantities of the composition are infused into a standard set of implants or coated onto or adsorbed to the surface of or both coated and infused, and the standard set of implants is implanted in vivo. This method could be used to measure levels of different growth factor in any tissue. For example, for osteogenesis, the combination of TGF-β1 (a mitogen) and BMP2/4 (a morphogen) would be identified and measured. Other combinations could be identified and measured, such as, for example, TGF-β1 and BMP-13 (CDMP, GDF-5) depending upon a particular interest.

### EXAMPLE 9

### Measurement of the Chondrogenic Capacity of a Bone Implant

The chondrogenic capacity of a bone implant is measured in vitro by releasing, chondrogenic factors from the implant (e.g. BMP-2, and BMP-4), in vitro measuring the concentrations of chondrogenic factors present, and exposing tissue containing mesenchymal or other undifferentiated cells to a composition of these proteins. The degree of development of chondroblasts and chondrocytes in vitro is used to confirm the chondrogenic capacity of the implant predicted by the present in vitro assay method. The degree of differentiation can be manipulated to reach a desired result by altering the specific concentrations of chondrogenic factors included in a given implant.

### EXAMPLE 10

### Method of Using Compositions in Wound Healing

Wound healing and the rate of recovery from bone damage or disease may be accelerated by applying a therapeutic composition of BMP and/or TGF to a site. As these proteins play varying regulatory roles in the healing process, depending on the type of injury presented, a composition of proteins is designed that contains a therapeutic quantity of one or more of these proteins. Utilizing the methodology of the present invention, direct quantitation of the total quantity of factor to be used to achieve a given in vivo result is reliably predicted. Additionally, a combination of a morphogen with a mitogen may be developed such that the combination yields more of the desired tissue than either alone. For example, the product of BMP-2/4 and TGFβ1 provide better osteoinductivity than when either component is used separately.

### EXAMPLE 11

### Method of Using Assay Results for Prognosis and Treatment of Cancer

A biopsy of bone or other tissue is taken from a patient. Bone proteins known to be active in cancer development maintenance or destruction (e.g. BMP-2, 4 TGF-β1) are isolated, purified and quantified in vitro. Concentration of the proteins are then used to assess the type of malignancy (e.g. for bone, whether a carcinoma is osteolytic or osteogenic) and treatment is adjusted accordingly. Those skilled in the art will appreciate in view of the teachings herein that the subject methods can be readily modified to analyze other types of cancers, including lung, breast, prostate and others. As the concentration of certain proteins present in a given tissue or fluid has been linked to cancerous activity, the present invention provides a fast, simple assay that is used for the accurate diagnosis of cancer.

### EXAMPLE 12

### Method for Using Assay Results in Prognosis and Treatment of Developmental Disorders

The in vitro quantification of bone proteins present in a given tissue at various stages of development is measured. By comparing the quantity of proteins known to act in cell proliferation, apoptosis, differentiation and morphogenesis present at a certain developmental stage, to normal baseline values, the causes of neurological, skeletal, developmental and other disorders is elucidated. Appropriate treatment regimens can then be established.

### EXAMPLE 13

### An in vitro Method for Determining Whether a Substance Will be Osteoinductive Prior to Implantation

The in vitro assay of the present invention allows a user to quantify the osteoinductive capacity of an implantable material, prior to implantation and therefore eliminates the need for live animal testing prior to human implantation. In a further embodiment according this invention, however, the inverse applies. That is, in a given implant, where it is desirable to confirm that a given implant will not induce bone formation upon implantation. According to this embodiment of the invention, an implant is assayed in vitro for as many specific osteogenic factors as are considered relevant to the given implant type to ensure that the implant will not induce new bone formation upon implantation. Thus, for example, with respect to a demineralized bone implant which is used as a ligament replacement, it is desirable to be sure that there is minimal or no new bone formation in the flexible portion of the bone implant. Accordingly, that portion of the implant, or a representative sampling of implants from an inventory of implants is assayed according to the method of this invention to ensure that there is less than a specified amount of BMP-2/4, TGF-β1, or other known osteoinductive factors, to ensure that upon implantation, the ligament will continue to operate as a ligament and will not ossify.

The disclosure of all patents and publications cited in this application are incorporated by reference in their entirety to the extent that their teachings are not inconsistent with the teachings herein. It should be understood that the examples and embodiments described herein are for illustrative purposes only and that various modifications or changes in light thereof will be suggested to persons skilled in the art and are to be included within the spirit and purview of this application and the scope of the appended claims.

What is claimed is:

1. A method for quantifying the osteoinductive potential of a collection of like implant material intended for implantation into human or non-human recipients in need thereof comprising:
(a) releasing osteogenic factors from a representative sampling of a collection of like implant materials to produce an implant releasate containing said osteogenic factors; and
(b) quantifying the concentration of at least one osteogenic factor present in said implant releasate, wherein said quantifying occurs in vitro and does not require implantation of said materials in vivo or use of complex biological living materials; and
(c) determining a value of osteogenic potential for said representative sampling by corresponding said concentration of at least one osteogenic factor with a similar value on a predetermined curve;
whereby the osteogenic potential of said collection is realized.

2. The method according to claim 1, wherein said implant material comprises bone.

3. The method according to claim 2, wherein said bone implant material comprises autograft, allograft, xenograft, cortical bone, cancellous bone, and combinations thereof.

4. The method according to claim 3, wherein said releasing of step (a) comprises demineralizing bone implant material to produce a substantially demineralized bone implant matrix; optionally said demineralizing bone implant material comprises reducing calcium concentration to about 2 percent or less.

5. The method according to claim 4, wherein said releasing further comprises dissolving said demineralized bone implant matrix.

6. The method according to claim 5, wherein said dissolving comprises contacting said demineralized bone implant matrix with enzymes that do not destroy osteoinductive factors present in said implant releasate, but which dissolve or otherwise dissociate said demineralized bone matrix to produce a dissolved implant releasate.

7. The method according to claim 6, wherein said enzymes comprise collagenase.

8. The method according to claim 6, wherein said method further comprises removing particulate debris from said dissolved implant releasate.

9. The method according to claim 8, wherein said removing comprises centrifuging said dissolved implant releasate and retaining the centrifugation supernatant to provide an implant releasate supernatant.

10. The method according to claim 9 further comprising removing low molecular weight non-osteogenic factor molecules from said implant releasate supernatant.

11. The method according to claim 10, wherein said removing low molecular weight non-osteogenic factor molecules comprises subjecting said implant releasate to dialyzing, ultrafiltering, size-exclusion fractionating, precipitating, or combinations thereof.

12. The method according to claim 1, wherein said at least one osteogenic factor comprises at least one mitogen and at least one morphogen.

13. The method according to claim 1, wherein said at least one osteoinductive factor is selected from the group consisting of bone morphogenetic proteins, tissue growth factors, fibroblast growth factors, platelet derived growth factors, vascular endothelial growth factors, cartilage derived morphogenetic proteins, insulin-like growth factors, and combinations thereof.

14. The method according to claim 1, wherein said at least one osteogenic factor is selected from the group consisting of transforming growth factors TGF-α, TGF-β, bone morphogenic protein BMP-1, BMP-2, BMP-3, BMP-4, BMP-5, BMP-6, BMP-7, BMP-8 and combinations thereof.

15. The method according to claim 14 wherein said at least one osteogenic factor comprises TGF-β1 plus BMP-2 or BMP-4 or both.

16. The method according to claim 1, wherein said quantifying comprises utilizing an immunoassay which detects specific osteoinductive factors present in said implant releasate.

17. The method according to claim 16, wherein said immunoassay is selected from the group consisting of enzyme-linked immunosorbent assay (ELIZA), radioimmunoassay, immunoprecipitation or combinations thereof.

18. The method according to claim 16 wherein said quantifying comprises contacting said at least one osteogenic factor with an antibody specific thereto under conditions to allow for specific binding of said antibody to said at least one osteogenic factor to occur, and measuring said specific binding of said antibody to said at least one osteogenic factor.

19. The method according to claim 1, wherein said osteoinductive factors are quantified in the range between picogram and milligram quantities and multiples and dilutions thereof.

20. The method according to claim 1, wherein said predetermined curve is established by correlating concentrations of at least one osteogenic factor with the probability of said concentrations to generate bone in vivo.

21. The method according to claim 20, wherein said correlating concentrations of at least one osteogenic factor comprises correlating the product achieved by multiplying a given concencentration of TGF-β1 with a concentration of BMP2, BMP4 or both.

22. The method according to claim 1 wherein said predetermined curve is established by correlating concentration of at least one osteogenic factor with an ability to induce differentitation of undifferentiated cells.

23. A method of measuring the osteogenic potential of an implant comprising:
(a) releasing osteogenic factors from said implant to produce an implant releasate containing said osteogenic factors;
(b) quantifying the concentration of at least one osteogenic factor in said implant releasate, wherein said quantifying occurs in vitro and does not require implantation of said implant in vivo or use of complex biological living materials; and
(c) determining a value of osteogenic potential for said implant by corresponding said concentration of at least one osteogenic factor with a similar value on a predetermined curve;
whereby the osteogenenic potential of said implant is realized.

24. A method of measuring the chondrogenic capacity of an implant comprising:
(a) releasing chondrogenic factors from said implant to produce an implant releasate containing said chondrogenic factors;
(b) quantifying the concentration of at least one chondrogenic factor in said implant releasate, wherein said quantifying occurs in vitro and does not require implantation of said implant in vivo or use of complex biological living materials; and
(c) determining a value of chondrogenic capacity for said implant by corresponding said concentration of at least one chondrogenic factor with a similar value on a predetermined curve;
whereby the chondrogenic capacity of said implant is realized.

25. A method of accelerating wound healing or the rate of recovery from bone damage or disease in a human or non-human patient in need thereof comprising:
(a) producing a composition comprising an amount of two or more growth factors, wherein said amount effects enhanced healing over a composition comprising just one of said two or more growth factors; and
(b) administering said composition to a patient in need thereof.

26. The method according to claim 25, wherein said amount is determined by employing the method of claim 1.

27. A method for the diagnosis and treatment of bone or soft-tissue cancer in a human or non-human patient in need thereof comprising:
(a) harvesting bone or soft-tissue from a donor;
(b) isolating and purifying osteogenic material therefrom; and
(c) comparing the quantity and type of growth factors present to that found in healthy bone or other tissues.

28. A method for assessing developmental bone or tissue disorders comprising:
(a) harvesting a bone or soft-tissue sample from a selected area at different stages of development;
(b) isolating, purifying and quantifying the osteogenic factors present in said sample;
(c) comparing the quantity and type of osteogenic factors present at different stages of the development of said bone or tissue with established baseline values; and
(d) identifying osteogenic factors present in elevated or decreased concentrations relative to said baseline value.

29. The method according to claim 27, further comprising formulating therapeutic compositions specific for counteracting said elevated or decreased concentrations of said osteogenic factors.

30. The method of claim 27, wherein said elevated or decreased level is associated with cellular proliferation, apoptosis, differentiation, morphogenesis or combinations thereof.

31. A method for reducing the need to sacrifice laboratory animals used in bone growth studies comprising selecting an implant, wherein the osteoinductive potential of said implant is predetermined by the method of claim 1; and implanting said implant into a patient in need thereof.

32. An implant selected from a collection of like implants, wherein the osteoinductive potential of said collection of like implants is predetermined by the method of claim 1.

33. A collection of like implants, wherein the osteoinductive potential of said collection of like implants is quantified by the method of claim 1, and wherein said collection of like implants is labelled as possessing osteoinductive potential as determined by the method of claim 1.

34. A composition for administration to a site of need comprising an admixture of at least one mitogenic factor and at least one morphogenic factor; wherein said composition is adapted such that upon administration of said composition, an amount of said morphogenic factor is released after an amount of said mitogenic factor is released.

35. The composition of claim 34, wherein said mitogenic factor is TGF-beta and said morphogenic factor is BMP-2.

36. The composition of claim 34, wherein said admixture is provided in a pharmaceutically acceptable carrier.
